(19) United States
(12) Patent Application Publication
(10) Pub. No.: US 2024/0183667 A1
(43) Pub. Date: Jun. 6, 2024

(54) USING IOT AND ANALYTICS TO PRIORITIZE DISPATCH OF MEDICAL SUPPLIES BY DYNAMIC ROUTING OF AUTONOMOUS VEHICLES

(71) Applicant: INTERNATIONAL BUSINESS MACHINES CORPORATION, Armonk, NY (US)

(72) Inventors: Jasbir Singh Dhaliwal, Noida (IN); Sanket Jain, Gurgaon (IN); Jatinder S. Joshi, Gurgaon (IN); Vivek Dabas, Delhi (IN)

(21) Appl. No.: 18/072,930

(22) Filed: Dec. 1, 2022

(51) Int. Cl. G01C 21/34; G06Q 10/0835; G16H 40/00

(52) U.S. Cl. CPC G01C 21/3407; G01C 21/3453; G06Q 10/08355; G16H 40/00

(57) ABSTRACT

A computer-implemented method for efficiently dispatching one or more autonomous vehicles to deliver medical supplies to a destination. The method determines a level of emergency and a disease condition at the destination via a user communication interface on the one or more autonomous vehicles. The method prioritizes dispatching the one or more autonomous vehicles based on the level of emergency and the disease condition at the destination. The method optimizes a route of the one or more autonomous vehicles to the destination. The method further integrates a plurality of available data sources across multiple locations, compares these data sources, and builds deep learning (DL) forecasting models to predict several different medicines required, corresponding to a distribution of the disease condition, across the multiple locations.

### BACKGROUND

The present disclosure relates generally to the field of deep learning (DL) and more particularly to internet of things (IoT), analytics, and forecasting models in medical supply delivery via autonomous vehicles.

The ineffective distribution of medical supplies, as well as unavailability of medical staff to administer the medical supplies, to people who need them in their time of need can lead to disease-related consequences.

The Covid-19 pandemic has encouraged many people to be more proactive in demanding medication and treatment prior to developing a serious illness. However, ineffective dispatching of medications have led to waste and the inability to effectively treat a population that needs it the most.

Currently, there is no efficient forecasting model to prioritize and distribute medical supplies to affected locations via autonomous vehicles.

### BRIEF SUMMARY

Embodiments of the present invention disclose a method, a computer program product, and a system, for efficiently dispatching one or more autonomous vehicles to deliver medical supplies to a destination.

According to an embodiment, the method determines a level of emergency and a disease condition at the destination via a user communication interface on the one or more autonomous vehicles. The method further prioritizes dispatching the one or more autonomous vehicles based on the level of emergency and the disease condition at the destination. The method further optimizes a route of the one or more autonomous vehicles to the destination and builds deep learning (DL) forecasting models to predict several different medicines required, corresponding to a distribution of the disease condition, across multiple locations.

A computer program product, according to an embodiment of the invention, includes a non-transitory tangible storage device having program code embodied therewith. The program code is executable by a processor of a computer to perform a method. The method determines a level of emergency and a disease condition at the destination via a user communication interface on the one or more autonomous vehicles. The method further prioritizes dispatching the one or more autonomous vehicles based on the level of emergency and the disease condition at the destination. The method further optimizes a route of the one or more autonomous vehicles to the destination and builds deep learning (DL) forecasting models to predict several different medicines required, corresponding to a distribution of the disease condition, across multiple locations.

A computer system, according to an embodiment of the invention, includes one or more computer devices each having one or more processors and one or more tangible storage devices; and a program embodied on at least one of the one or more storage devices, the program having a plurality of program instructions for execution by the one or more processors. The program instructions implement a method. The method determines a level of emergency and a disease condition at the destination via a user communication interface on the one or more autonomous vehicles. The method further prioritizes dispatching the one or more autonomous vehicles based on the level of emergency and the disease condition at the destination. The method further optimizes a route of the one or more autonomous vehicles to the destination and builds deep learning (DL) forecasting models to predict several different medicines required, corresponding to a distribution of the disease condition, across multiple locations.

### BRIEF DESCRIPTION OF THE DRAWINGS

FIG. 1 depicts a diagram graphically illustrating the hardware components of autonomous vehicle prioritizer computing environment 200 and a cloud computing environment, in accordance with an embodiment of the present invention.

FIG. 2 illustrates autonomous vehicle prioritizer computing environment 200, in accordance with an embodiment of the present invention.

FIG. 3 is a flowchart illustrating the operation of autonomous vehicle prioritizer program 220 of FIG. 2, in accordance with an embodiment of the present invention.

### DETAILED DESCRIPTION

The present invention discloses a method for efficiently dispatching one or more autonomous vehicles, using IoT and advanced analytics, to deliver key medical supplies to a destination for patients who are in need.

The present disclosure does not just deliver medical supplies to a single needed destination, but additionally prioritizes deliveries based on the disease condition (e.g., Covid-19, etc.) and optimizing the route, or path, of each autonomous vehicle.

In exemplary embodiments, each autonomous vehicle is an unmanned aerial vehicle (UAV).

As such, the present invention provides a more effective and robust medical supply (e.g., vaccines, medications, etc.) distribution for infected populations.

This improvement in the field of autonomous vehicle technology and prioritized medical supply delivery can have a tremendous impact on the reduction of spread of infectious diseases and disease-related illnesses.

Nowadays, as the world is recovering from the Covid-19 pandemic, medical professionals see the importance of providing effective and available treatments to people in need, on a prioritized basis depending on disease condition and the number of people affected in a particular location.

Hereinafter, exemplary embodiments of the present invention will be described in detail with reference to the attached drawings.

The present invention is not limited to the exemplary embodiments below but may be implemented with various modifications within the scope of the present invention. In addition, the drawings used herein are for purposes of illustration, and may not show actual dimensions.

Various aspects of the present disclosure are described by narrative text, flowcharts, block diagrams of computer systems and/or block diagrams of the machine logic included in computer program product (CPP) embodiments. With respect to any flowcharts, depending upon the technology involved, the operations can be performed in a different order than what is shown in a given flowchart. For example, again depending upon the technology involved, two operations shown in successive flowchart blocks may be performed in reverse order, as a single integrated step, concurrently, or in a manner at least partially overlapping in time.

A computer program product embodiment (“CPP embodiment” or “CPP”) is a term used in the present disclosure to describe any set of one, or more, storage media (also called “mediums”) collectively included in a set of one, or more, storage devices that collectively include machine readable code corresponding to instructions and/or data for performing computer operations specified in a given CPP claim. A “storage device” is any tangible device that can retain and store instructions for use by a computer processor. Without limitation, the computer readable storage medium may be an electronic storage medium, a magnetic storage medium, an optical storage medium, an electromagnetic storage medium, a semiconductor storage medium, a mechanical storage medium, or any suitable combination of the foregoing. Some known types of storage devices that include these mediums include: diskette, hard disk, random access memory (RAM), read-only memory (ROM), erasable programmable read-only memory (EPROM or Flash memory), static random access memory (SRAM), compact disc read-only memory (CD-ROM), digital versatile disk (DVD), memory stick, floppy disk, mechanically encoded device (such as punch cards or pits/lands formed in a major surface of a disc) or any suitable combination of the foregoing. A computer readable storage medium, as that term is used in the present disclosure, is not to be construed as storage in the form of transitory signals per se, such as radio waves or other freely propagating electromagnetic waves, electromagnetic waves propagating through a waveguide, light pulses passing through a fiber optic cable, electrical signals communicated through a wire, and/or other transmission media. As will be understood by those of skill in the art, data is typically moved at some occasional points in time during normal operations of a storage device, such as during access, de-fragmentation or garbage collection, but this does not render the storage device as transitory because the data is not transitory while it is stored.

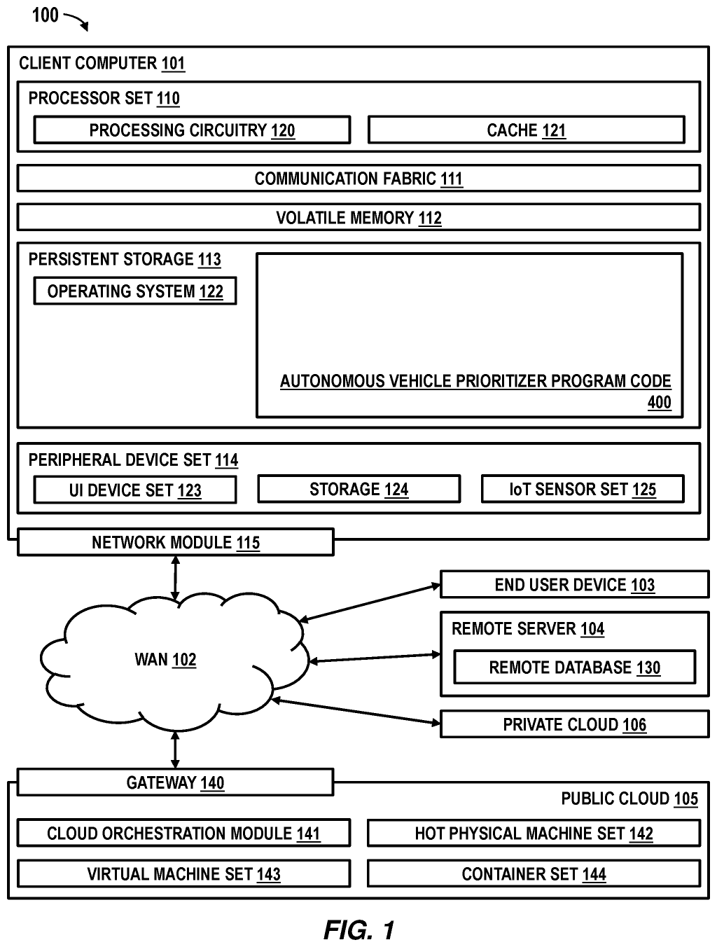

FIG. 1 depicts a diagram graphically illustrating the hardware components of autonomous vehicle prioritizer computing environment 200 and a cloud computing environment in accordance with an embodiment of the present invention.

Autonomous vehicle prioritizer computing environment 200 contains an example of an environment for the execution of at least some of the computer code involved in performing the inventive methods, such as autonomous vehicle prioritizer program code 400. In addition to autonomous vehicle prioritizer program code 400, computing environment 100 includes, for example, computer 101, wide area network (WAN) 102, end user device (EUD) 103, remote server 104, public cloud 105, and private cloud 106. In this embodiment, computer 101 includes processor set 110 (including processing circuitry 120 and cache 121), communication fabric 111, volatile memory 112, persistent storage 113 (including operating system 122 and autonomous vehicle prioritizer program code 400, as identified above), peripheral device set 114 (including user interface (UI), device set 123, storage 124, and Internet of Things (IoT) sensor set 125), and network module 115. Remote server 104 includes remote database 130. Public cloud 105 includes gateway 140, cloud orchestration module 141, host physical machine set 142, virtual machine set 143, and container set 144.

COMPUTER 101 may take the form of a desktop computer, laptop computer, tablet computer, smart phone, smart watch or other wearable computer, mainframe computer, quantum computer or any other form of computer or mobile device now known or to be developed in the future that is capable of running a program, accessing a network or querying a database, such as remote database 130. As is well understood in the art of computer technology, and depending upon the technology, performance of a computer-implemented method may be distributed among multiple computers and/or between multiple locations. On the other hand, in this presentation of computing environment 100, detailed discussion is focused on a single computer, specifically computer 101, to keep the presentation as simple as possible. Computer 101 may be located in a cloud, even though it is not shown in a cloud in FIG. 1. On the other hand, computer 101 is not required to be in a cloud except to any extent as may be affirmatively indicated.

PROCESSOR SET 110 includes one, or more, computer processors of any type now known or to be developed in the future. Processing circuitry 120 may be distributed over multiple packages, for example, multiple, coordinated integrated circuit chips. Processing circuitry 120 may implement multiple processor threads and/or multiple processor cores. Cache 121 is memory that is located in the processor chip package(s) and is typically used for data or code that should be available for rapid access by the threads or cores running on processor set 110. Cache memories are typically organized into multiple levels depending upon relative proximity to the processing circuitry. Alternatively, some, or all, of the cache for the processor set may be located “off chip.” In some computing environments, processor set 110 may be designed for working with qubits and performing quantum computing.

Computer readable program instructions are typically loaded onto computer 101 to cause a series of operational steps to be performed by processor set 110 of computer 101 and thereby effect a computer-implemented method, such that the instructions thus executed will instantiate the methods specified in flowcharts and/or narrative descriptions of computer-implemented methods included in this document (collectively referred to as “the inventive methods”). These computer readable program instructions are stored in various types of computer readable storage media, such as cache 121 and the other storage media discussed below. The program instructions, and associated data, are accessed by processor set 110 to control and direct performance of the inventive methods. In computing environment 100, at least some of the instructions for performing the inventive methods may be stored in autonomous vehicle prioritizer program code 400 in persistent storage 113.

COMMUNICATION FABRIC 111 is the signal conduction paths that allow the various components of computer 101 to communicate with each other. Typically, this fabric is made of switches and electrically conductive paths, such as the switches and electrically conductive paths that make up busses, bridges, physical input/output ports and the like. Other types of signal communication paths may be used, such as fiber optic communication paths and/or wireless communication paths.

VOLATILE MEMORY 112 is any type of volatile memory now known or to be developed in the future. Examples include dynamic type random access memory (RAM) or static type RAM. Typically, the volatile memory is characterized by random access, but this is not required unless affirmatively indicated. In computer 101, the volatile memory 112 is located in a single package and is internal to computer 101, but, alternatively or additionally, the volatile memory may be distributed over multiple packages and/or located externally with respect to computer 101.

PERSISTENT STORAGE 113 is any form of non-volatile storage for computers that is now known or to be developed in the future. The non-volatility of this storage means that the stored data is maintained regardless of whether power is being supplied to computer 101 and/or directly to persistent storage 113. Persistent storage 113 may be a read only memory (ROM), but typically at least a portion of the persistent storage allows writing of data, deletion of data and re-writing of data. Some familiar forms of persistent storage include magnetic disks and solid state storage devices. Operating system 122 may take several forms, such as various known proprietary operating systems or open-source Portable Operating System Interface type operating systems that employ a kernel. The code included in autonomous vehicle prioritizer program code 400 typically includes at least some of the computer code involved in performing the inventive methods.

PERIPHERAL DEVICE SET 114 includes the set of peripheral devices of computer 101. Data communication connections between the peripheral devices and the other components of computer 101 may be implemented in various ways, such as Bluetooth connections, Near-Field Communication (NFC) connections, connections made by cables (such as universal serial bus (USB) type cables), insertion type connections (for example, secure digital (SD) card), connections made though local area communication networks and even connections made through wide area networks such as the internet. In various embodiments, UI device set 123 may include components such as a display screen, speaker, microphone, wearable devices (such as goggles and smart watches), keyboard, mouse, printer, touchpad, game controllers, and haptic devices. Storage 124 is external storage, such as an external hard drive, or insertable storage, such as an SD card. Storage 124 may be persistent and/or volatile. In some embodiments, storage 124 may take the form of a quantum computing storage device for storing data in the form of qubits. In embodiments where computer 101 is required to have a large amount of storage (for example, where computer 101 locally stores and manages a large database) then this storage may be provided by peripheral storage devices designed for storing very large amounts of data, such as a storage area network (SAN) that is shared by multiple, geographically distributed computers. IoT sensor set 125 is made up of sensors that can be used in Internet of Things applications. For example, one sensor may be a thermometer and another sensor may be a motion detector.

NETWORK MODULE 115 is the collection of computer software, hardware, and firmware that allows computer 101 to communicate with other computers through WAN 102. Network module 115 may include hardware, such as modems or Wi-Fi signal transceivers, software for packetizing and/or de-packetizing data for communication network transmission, and/or web browser software for communicating data over the internet. In some embodiments, network control functions and network forwarding functions of network module 115 are performed on the same physical hardware device. In other embodiments (for example, embodiments that utilize software-defined networking (SDN)), the control functions and the forwarding functions of network module 115 are performed on physically separate devices, such that the control functions manage several different network hardware devices. Computer readable program instructions for performing the inventive methods can typically be downloaded to computer 101 from an external computer or external storage device through a network adapter card or network interface included in network module 115.

WAN 102 is any wide area network (for example, the internet) capable of communicating computer data over non-local distances by any technology for communicating computer data, now known or to be developed in the future. In some embodiments, the WAN may be replaced and/or supplemented by local area networks (LANs) designed to communicate data between devices located in a local area, such as a Wi-Fi network. The WAN and/or LANs typically include computer hardware such as copper transmission cables, optical transmission fibers, wireless transmission, routers, firewalls, switches, gateway computers and edge servers.

END USER DEVICE (EUD) 103 is any computer system that is used and controlled by an end user (for example, a customer of an enterprise that operates computer 101), and may take any of the forms discussed above in connection with computer 101. EUD 103 typically receives helpful and useful data from the operations of computer 101. For example, in a hypothetical case where computer 101 is designed to provide a recommendation to an end user, this recommendation would typically be communicated from network module 115 of computer 101 through WAN 102 to EUD 103. In this way, EUD 103 can display, or otherwise present, the recommendation to an end user. In some embodiments, EUD 103 may be a client device, such as thin client, heavy client, mainframe computer, desktop computer and so on.

REMOTE SERVER 104 is any computer system that serves at least some data and/or functionality to computer 101. Remote server 104 may be controlled and used by the same entity that operates computer 101. Remote server 104 represents the machine(s) that collect and store helpful and useful data for use by other computers, such as computer 101. For example, in a hypothetical case where computer 101 is designed and programmed to provide a recommendation based on historical data, then this historical data may be provided to computer 101 from remote database 130 of remote server 104.

PUBLIC CLOUD 105 is any computer system available for use by multiple entities that provides on-demand availability of computer system resources and/or other computer capabilities, especially data storage (cloud storage) and computing power, without direct active management by the user. Cloud computing typically leverages sharing of resources to achieve coherence and economies of scale. The direct and active management of the computing resources of public cloud 105 is performed by the computer hardware and/or software of cloud orchestration module 141. The computing resources provided by public cloud 105 are typically implemented by virtual computing environments that run on various computers making up the computers of host physical machine set 142, which is the universe of physical computers in and/or available to public cloud 105. The virtual computing environments (VCEs) typically take the form of virtual machines from virtual machine set 143 and/or containers from container set 144. It is understood that these VCEs may be stored as images and may be transferred among and between the various physical machine hosts, either as images or after instantiation of the VCE. Cloud orchestration module 141 manages the transfer and storage of images, deploys new instantiations of VCEs and manages active instantiations of VCE deployments. Gateway 140 is the collection of computer software, hardware, and firmware that allows public cloud 105 to communicate through WAN 102.

Some further explanation of virtualized computing environments (VCEs) will now be provided. VCEs can be stored as “images.” A new active instance of the VCE can be instantiated from the image. Two familiar types of VCEs are virtual machines and containers. A container is a VCE that uses operating-system-level virtualization. This refers to an operating system feature in which the kernel allows the existence of multiple isolated user-space instances, called containers. These isolated user-space instances typically behave as real computers from the point of view of programs running in them. A computer program running on an ordinary operating system can utilize all resources of that computer, such as connected devices, files and folders, network shares, CPU power, and quantifiable hardware capabilities. However, programs running inside a container can only use the contents of the container and devices assigned to the container, a feature which is known as containerization.

PRIVATE CLOUD 106 is similar to public cloud 105, except that the computing resources are only available for use by a single enterprise. While private cloud 106 is depicted as being in communication with WAN 102, in other embodiments a private cloud may be disconnected from the internet entirely and only accessible through a local/private network. A hybrid cloud is a composition of multiple clouds of different types (for example, private, community or public cloud types), often respectively implemented by different vendors. Each of the multiple clouds remains a separate and discrete entity, but the larger hybrid cloud architecture is bound together by standardized or proprietary technology that enables orchestration, management, and/or data/application portability between the multiple constituent clouds. In this embodiment, public cloud 105 and private cloud 106 are both part of a larger hybrid cloud.

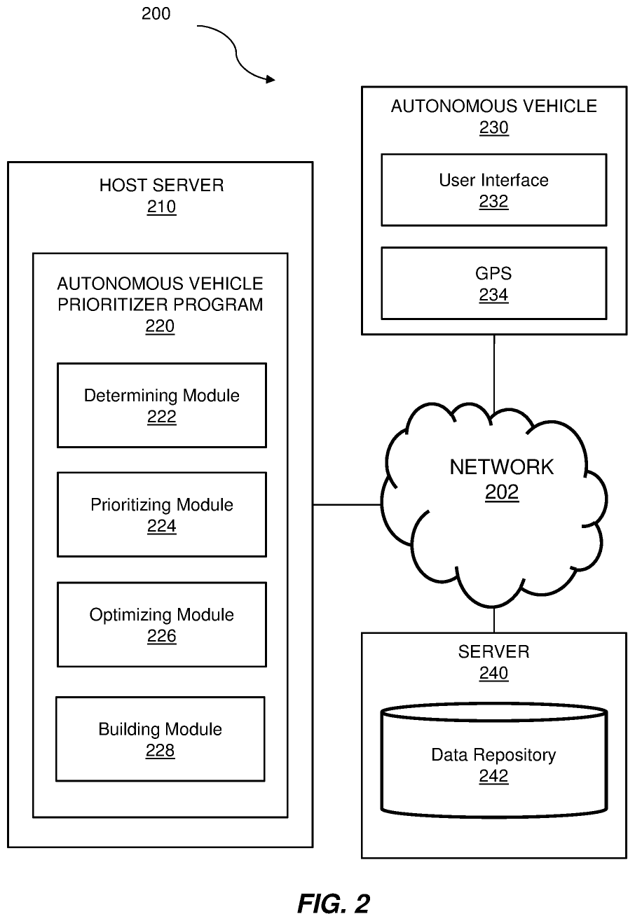

FIG. 2 illustrates autonomous vehicle prioritizer computing environment 200, in accordance with an embodiment of the present invention. Autonomous vehicle prioritizer computing environment 200 includes host server 210, autonomous vehicle 230, and server 240 all connected via network 202. The setup in FIG. 2 represents an example embodiment configuration for the present invention and is not limited to the depicted setup to derive benefit from the present invention.

In an exemplary embodiment, host server 210 includes autonomous vehicle prioritizer program 220. In various embodiments, host server 210 may be a laptop computer, tablet computer, netbook computer, personal computer (PC), a desktop computer, a personal digital assistant (PDA), a smart phone, or any programmable electronic device capable of communicating with autonomous vehicle 230 and server 240 via network 202. Host server 210 may include internal and external hardware components, as depicted, and described in further detail with reference to FIG. 1. In other embodiments, host server 210 may be implemented in a cloud computing environment, as further described in relation to FIG. 1 herein. Host server 210 may also have wireless connectivity capabilities allowing it to communicate with autonomous vehicle 230, server 240, and other computers or servers over network 202.

With continued reference to FIG. 2, autonomous vehicle 230 includes user interface 232 and global positioning system (GPS) 234 and may be a laptop computer, tablet computer, netbook computer, personal computer (PC), a desktop computer, a personal digital assistant (PDA), a smart phone, or any programmable electronic device capable of communicating with host server 210 and server 240 via network 202. Autonomous vehicle 230 may include internal and external hardware components, as depicted, and described in further detail below with reference to FIG. 1. In other embodiments, autonomous vehicle 230 may be implemented in a cloud computing environment, as described in relation to FIG. 1, herein.

In exemplary embodiments, user interface 232 is a computer program which allows a user to interact with autonomous vehicle 230 and other connected devices via network 202. For example, user interface 232 may be a graphical user interface (GUI). In addition to comprising a computer program, user interface 232 may be connectively coupled to hardware components, such as those depicted in FIG. 1, for receiving user input. In an exemplary embodiment, user interface 232 may be a web browser, however in other embodiments user interface 232 may be a different program capable of receiving user interaction and communicating with other devices.

In exemplary embodiments, user interface 232 may include a user communication interface (e.g., a chatbot, a smart speaker, voice commands, keyboard, etc.) that utilizes artificial intelligence (AI). The user communication interface may also display results based on determining the level of emergency and the kind of disease or ailment that a patient is suffering from.

In exemplary embodiments, GPS 234 is a computer program on autonomous vehicle 230 that provides time and location information for a user. Modern GPS systems operate on the concept of time and location. In modern GPS systems, four or more satellites broadcast a continuous signal detailing satellite identification information, time of transmission (TOT), and the precise location of the satellite at the time of transmission. When a GPS receiver picks up the signal, it determines the difference in time between the time of transmission (TOT) and the time of arrival (TOA). Based on the amount of time it took to receive the signals and the precise locations of the satellites when the signals were sent, GPS receivers can determine the location where the signals were received. In the exemplary embodiment, GPS 234 can provide real-time location detection of autonomous vehicle 230, together with an estimated time of arrival for a given destination. GPS 234 may also include alternate routes and/or means of transportation to reach a destination.

In exemplary embodiments, GPS 234 may include real-time information regarding emergency happenings on a route that may affect estimated travel time information. In various embodiments, GPS 234 may provide alternate routes to reach a destination.

With continued reference to FIG. 2, server 240 comprises data repository 242 and may be a laptop computer, tablet computer, netbook computer, personal computer (PC), a desktop computer, a personal digital assistant (PDA), a smart phone, or any programmable electronic device capable of communicating with host server 210 and autonomous vehicle 230 via network 202.

In exemplary embodiments, data repository 242 stores knowledge of autonomous vehicles by city, population, disease commonality within a particular region/location, disease severity, dispatch priority, and any other metric that is useful for autonomous vehicle prioritizer program 220 to assess disease commonality.

With continued reference to FIG. 2, host server 210 includes autonomous vehicle prioritizer program 220. Host server 210 may be a laptop computer, tablet computer, netbook computer, personal computer (PC), a desktop computer, a personal digital assistant (PDA), a smart phone, or any programmable electronic device capable of communicating with autonomous vehicle 230 and server 240 via network 202.

With continued reference to FIG. 2, autonomous vehicle prioritizer program 220, in an exemplary embodiment, may be a computer application on host server 210 that contains instruction sets, executable by a processor. The instruction sets may be described using a set of functional modules. In exemplary embodiments, autonomous vehicle prioritizer program 220 may receive input from autonomous vehicle 230 and server 240 over network 202. In alternative embodiments, autonomous vehicle prioritizer program 220 may be a computer application on autonomous vehicle 230, or a standalone program on a separate electronic device.

With continued reference to FIG. 2, the functional modules of autonomous vehicle prioritizer program 220 include determining module 222, prioritizing module 224, optimizing module 226, and building module 228.

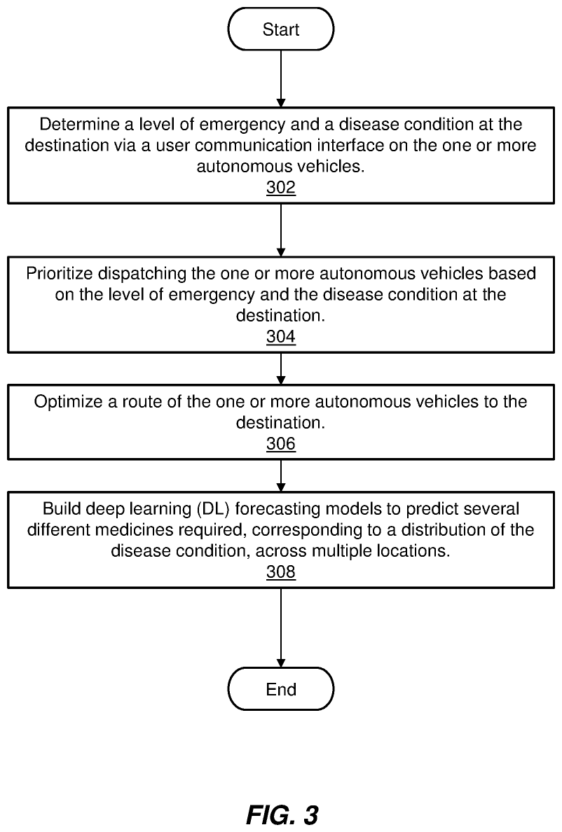

FIG. 3 is a flowchart illustrating the operation of autonomous vehicle prioritizer program 220 of FIG. 2, in accordance with embodiments of the present disclosure.

With reference to FIGS. 2 and 3, determining module 222 includes a set of programming instructions, in autonomous vehicle prioritizer program 220, to determine a level of emergency and a disease condition at the destination via a user communication interface on the one or more autonomous vehicles (step 302). The set of programming instructions is executable by a processor.

In exemplary embodiments, determining module 222 identifies patients who need medical supplies (e.g., Covid-19 related vaccines or other vaccines/medications) or who have recently suffered an ailment or symptom (e.g., broken leg, poor memory) that requires attention.

In alternative embodiments, determining module 222 can identify which geographical regions need medical supplies, or key medical products, and support staff.

The Covid-19 pandemic has arguably encouraged customers (i.e., patients) to seek medicines and treatment in a proactive manner rather than a reactive manner. This invention seeks to ensure the fulfillment of sought-after treatments, for both Covid-19 as well as severe diseases that may have arisen as a result of Covid-19, such as heart attack, stroke, diabetes, yellow fungus, high blood pressure, cholesterol, etc. Additionally, the current invention seeks to ensure that most of the customers who want to get tested for Covid-19 and get treated for same can do so while being at home.

With reference to an illustrative example, Joe has observed a contagious measles outbreak in his community. Determining module 222, via the use of an AI conversational interface on Joe's smartphone, can determine the level of emergency and the kind of disease outbreak based on Joe's description of what is happening around him. In another part of town, Walter is reporting, via determining module 222, on his smartphone, many residents suffering from pneumonia.

With continued reference to FIGS. 2 and 3, prioritizing module 224 includes a set of programming instructions in autonomous vehicle prioritizer program 220, to prioritize dispatching the one or more autonomous vehicles based on the level of emergency and the disease condition at the destination (step 304). The set of programming instructions is executable by a processor.

In exemplary embodiments, prioritizing module 224 utilizes IoT and advanced analytics to prioritize the dispatch of key medical supplies for patients who are in need, by using one or more autonomous vehicles, such as autonomous vehicle 230.

In further exemplary embodiments, prioritizing module 224 is capable of clustering cities, and streets within cities, based on a distribution of similar kinds of ailments/diseases. For example, X % of candidate patients in zip code 12345 of city C1 are suffering from Covid-19 for the first time, Y % of candidate patients are suffering from a heart attack due to a recent Covid-19 infection, Z % are suffering from another disorder (e.g., Autism, ADHD, memory loss, sleep walking, Down Syndrome, etc.), T % have severe depression, etc.

Additionally, prioritizing module 224 may prioritize the dispatch of the one or more autonomous vehicles based on the distribution of the disease condition.

In exemplary embodiments, prioritizing module 224 can further schedule an arrival of medical staff in conjunction with the delivery of the medical supplies to the destination via the dispatched one or more autonomous vehicles.

Autonomous vehicle prioritizer program 220 prioritizes dispatching logic based on a determined disease condition and optimizing the route/path of each UAV to ensure that the customers (i.e., patients) receive treatment to as much of an extent as possible.

With continued reference to the illustrative example above, prioritizing module 224 prioritizes a autonomous vehicle with needed medical supplies (e.g., measles vaccines) to Joe's community to stop the spread of the measles outbreak, since Joe's community is an area with a high expected disease incidence. Autonomous vehicle prioritizer program 220 dispatches autonomous vehicles carrying measles vaccines, from a centralized location outside Joe's community, Point A, to a centralized location within Joe's community, Point B. To achieve the above, autonomous vehicle prioritizer program 220 identifies a certain number of people (e.g., 500 people) in Joe's community who need the vaccine. Based on this calculation, a set number of vaccines are sent to the centralized location in Joe's community, and nurses may subsequently be sent to administer the vaccine injections.

With continued reference to FIGS. 2 and 3, optimizing module 236 includes a set of programming instructions in autonomous vehicle prioritizer program 220, to optimize a route of the one or more autonomous vehicles to the destination (step 306). The set of programming instructions is executable by a processor.

In exemplary embodiments, optimizing module 236 dynamically routes and re-routes the route of the one or more autonomous vehicles by utilizing natural language processing (NLP), IoT, and analytics to efficiently dispatch the one or more autonomous vehicles. Furthermore, optimizing module 236 stores the knowledge of the one or more autonomous vehicles in a centralized region for each city (e.g., destination).

In further exemplary embodiments, optimizing module 236 enables the payload as a source for insights to reroute the one or more autonomous vehicles to save time required for the one or more autonomous vehicles to reach an urgently needed destination.

With continued reference to FIGS. 2 and 3, building module 238 includes a set of programming instructions in autonomous vehicle prioritizer program 220, to build deep learning (DL) forecasting models to predict several different medicines required, corresponding to a distribution of the disease condition, across the multiple locations (step 308). The set of programming instructions is executable by a processor.

In exemplary embodiments, building module 238 integrates a plurality of available data sources across multiple locations and compares the plurality of available data sources across the multiple locations. The plurality of available data sources across the multiple locations comprise: a medical stock keeping unit (SKU) of various disease condition vaccinations, and a level of severity of other disease conditions or accidents.

In exemplary embodiments, DL forecasting models comprise a Long Short-Term Memory (LSTM) model and an Exponential Smoothing Model and Recurrent Neural Network (ESRNN) model.

In various exemplary embodiments, forecasting models are used to rapidly evaluate the safety and efficacy of vaccine candidates (e.g. Covid-19 vaccine and other vaccine candidates), prioritize vaccine dispatch in areas with highly expected disease incidence, as well as help patients who are suffering the most.

Furthermore, mathematical, and statistical forecast models can inform the process of site selection, integrating available data sources and facilitating comparisons across multiple locations.

DL models, such as LSTM and ESRNN, are utilized for predicting the number of different medicines required corresponding to the number of infection cases (e.g., Covid-19 positive) in each location.

These types of data-driven models can support the implementation of flexible dispatch of medical supplies tailored to a particular pandemic or outbreak (e.g., Covid-19) or other key ailments such as diabetes and other illnesses that may have arisen due to Covid-19.

In exemplary embodiments, the LSTM model can learn a function that maps a sequence of past observations as input to an output observation. As such, the sequence of observations must be transformed into multiple examples from which the LSTM can learn.

In exemplary embodiments, the ESRNN model cleverly combines the classic Exponential Smoothing model (ES) and a Recurrent Neural Network (RNN). The ES decomposes the time series in level, trend, and seasonality components. The RNN is trained with all the series, has shared parameters and it is used to learn common local trends among the series. The ES parameters, on the other hand, are specific for each time series.

The application of DL models to identify which regions to send key medical supplies and medical staff, on a prioritized basis, provides for a more effective vaccine distribution in times of need (e.g., Covid-19 and other pandemics, etc.). Furthermore, DL models can study the impact of such effective vaccine distribution on the reduction of Covid-19, or other illness or disease, related fallout (e.g., heart attacks, diabetes, and other related illnesses).

With continued reference to the illustrative example above, the application of DL forecasting models identifies Joe's community as a prioritized location for the delivery of medical supplies and key medical vaccines to be delivered. Stopping the measles outbreak in its tracks is paramount. Walter's community also receives medical supplies to treat its pneumonia patients. However, Walter's community is not forecasted as the priority when compared to the measles outbreak. With the addition of autonomous vehicle dispatch locations, both Joe's and Walter's communities may receive their medical supplies simultaneously.

In exemplary embodiments, network 202 is a communication channel capable of transferring data between connected devices and may be a telecommunications network used to facilitate telephone calls between two or more parties comprising a landline network, a wireless network, a closed network, a satellite network, or any combination thereof. In another embodiment, network 202 may be the Internet, representing a worldwide collection of networks and gateways to support communications between devices connected to the Internet. In this other embodiment, network 202 may include, for example, wired, wireless, or fiber optic connections which may be implemented as an intranet network, a local area network (LAN), a wide area network (WAN), or any combination thereof. In further embodiments, network 202 may be a Bluetooth network, a WiFi network, or a combination thereof. In general, network 202 can be any combination of connections and protocols that will support communications between host server 210, autonomous vehicle 230, and server 240.

1. A computer-implemented method for efficiently dispatching one or more autonomous vehicles to deliver medical supplies to a destination, comprising:
determining a level of emergency and a disease condition at the destination via a user communication interface on the one or more autonomous vehicles;
prioritizing dispatching the one or more autonomous vehicles based on the level of emergency and the disease condition at the destination;
optimizing a route of the one or more autonomous vehicles to the destination;
integrating a plurality of available data sources across multiple locations;
comparing the plurality of available data sources across the multiple locations; and
building deep learning (DL) forecasting models to predict several different medicines required, corresponding to a distribution of the disease condition, across the multiple locations.

2. The computer-implemented method of claim 1, wherein optimizing a route of the one or more autonomous vehicles to the destination further comprises:
dynamically routing and re-routing the route of the one or more autonomous vehicles by utilizing natural language processing (NLP), Internet of Things (IoT), and analytics to efficiently dispatch the one or more autonomous vehicles; and
storing knowledge of the one or more autonomous vehicles in a centralized region.

3. The computer-implemented method of claim 2, further comprising:
clustering cities, and streets within the cities, based on a distribution of the disease condition; and
prioritizing the dispatch of the one or more autonomous vehicles based on the distribution of the disease condition.

4. The computer-implemented method of claim 1, further comprising:
enabling payload as a source for insights to reroute the one or more autonomous vehicles to save time required for the one or more autonomous vehicles to reach an urgently needed destination.

5. The computer-implemented method of claim 1, wherein the DL forecasting models comprise a Long Short-Term Memory (LSTM) model and an Exponential Smoothing Model and Recurrent Neural Network (ESRNN) model.

6. The computer-implemented method of claim 1, wherein the plurality of available data sources across multiple locations comprises: a medical stock keeping unit (SKU) of various disease condition vaccinations, and a level of severity of other disease conditions or accidents.

7. The computer-implemented method of claim 1, further comprising:
scheduling an arrival of medical staff in conjunction with the delivery of the medical supplies to the destination via the dispatched one or more autonomous vehicles.

8. A computer program product for implementing a program that manages a device, comprising a non-transitory tangible storage device having program code embodied therewith, the program code executable by a processor of a computer to perform a method, the method comprising:
determining a level of emergency and a disease condition at the destination via a user communication interface on the one or more autonomous vehicles;
prioritizing dispatching the one or more autonomous vehicles based on the level of emergency and the disease condition at the destination;
optimizing a route of the one or more autonomous vehicles to the destination;
integrating a plurality of available data sources across multiple locations;
comparing the plurality of available data sources across the multiple locations; and
building deep learning (DL) forecasting models to predict several different medicines required, corresponding to a distribution of the disease condition, across the multiple locations.

9. The computer program product of claim 8, wherein optimizing a route of the one or more autonomous vehicles to the destination further comprises:
dynamically routing and re-routing the route of the one or more autonomous vehicles by utilizing natural language processing (NLP), Internet of Things (IoT), and analytics to efficiently dispatch the one or more autonomous vehicles; and
storing knowledge of the one or more autonomous vehicles in a centralized region.

10. The computer program product of claim 9, further comprising:
clustering cities, and streets within the cities, based on a distribution of the disease condition; and
prioritizing the dispatch of the one or more autonomous vehicles based on the distribution of the disease condition.

11. The computer program product of claim 8, further comprising:
enabling payload as a source for insights to reroute the one or more autonomous vehicles to save time required for the one or more autonomous vehicles to reach an urgently needed destination.

12. The computer program product of claim 8, wherein the DL forecasting models comprise a Long Short-Term Memory (LSTM) model and an Exponential Smoothing Model and Recurrent Neural Network (ESRNN) model.

13. The computer program product of claim 8, wherein the plurality of available data sources across multiple locations comprises: a medical stock keeping unit (SKU) of various disease condition vaccinations, and a level of severity of other disease conditions or accidents.

14. The computer program product of claim 8, further comprising:
scheduling an arrival of medical staff in conjunction with the delivery of the medical supplies to the destination via the dispatched one or more autonomous vehicles.

15. A computer system for implementing a program that manages a device, comprising:
one or more computer devices each having one or more processors and one or more tangible storage devices; and a program embodied on at least one of the one or more storage devices, the program having a plurality of program instructions for execution by the one or more processors, the program instructions comprising instructions for:
determining a level of emergency and a disease condition at the destination via a user communication interface on the one or more autonomous vehicles;
prioritizing dispatching the one or more autonomous vehicles based on the level of emergency and the disease condition at the destination;
optimizing a route of the one or more autonomous vehicles to the destination;
integrating a plurality of available data sources across multiple locations;
comparing the plurality of available data sources across the multiple locations; and
building deep learning (DL) forecasting models to predict several different medicines required, corresponding to a distribution of the disease condition, across the multiple locations.

16. The computer system of claim 15, wherein optimizing a route of the one or more autonomous vehicles to the destination further comprises:
dynamically routing and re-routing the route of the one or more autonomous vehicles by utilizing natural language processing (NLP), Internet of Things (IoT), and analytics to efficiently dispatch the one or more autonomous vehicles; and
storing knowledge of the one or more autonomous vehicles in a centralized region.

17. The computer system of claim 16, further comprising:
clustering cities, and streets within the cities, based on a distribution of the disease condition; and
prioritizing the dispatch of the one or more autonomous vehicles based on the distribution of the disease condition.

18. The computer system of claim 15, further comprising:
enabling payload as a source for insights to reroute the one or more autonomous vehicles to save time required for the one or more autonomous vehicles to reach an urgently needed destination.

19. The computer system of claim 15, wherein the DL forecasting models comprise a Long Short-Term Memory (LSTM) model and an Exponential Smoothing Model and Recurrent Neural Network (ESRNN) model.

20. The computer system of claim 15, wherein the plurality of available data sources across multiple locations comprises: a medical stock keeping unit (SKU) of various disease condition vaccinations, and a level of severity of other disease conditions or accidents.
